(19) United States
(12) Patent Application Publication
(10) Pub. No.: US 2021/0196944 A1
(43) Pub. Date: Jul. 1, 2021

(54) BLADDER URINE VOLUME MONITORING SYSTEM AND METHOD

(71) Applicant: Industrial Technology Research Institute, Hsinchu (TW)

(72) Inventors: Yi-Hsin Lin, Yunlin County (TW); Chia-Pin Li, Taichung City (TW); Kun-Ta Wu, Nantou County (TW); Kuo-Chun Lee, Hsinchu County (TW); Chun-Jung Chen, Hsinchu County (TW); Chun-Yu Chan, Hsinchu City (TW)

(73) Assignee: Industrial Technology Research Institute, Hsinchu (TW)

(21) Appl. No.: 16/907,319

(22) Filed: Jun. 22, 2020

Related U.S. Application Data
(60) Provisional application No. 62/954,631, filed on Dec. 29, 2019.

(30) Foreign Application Priority Data
May 8, 2020 (TW) 109115361

(51) Int. Cl. A61N 1/04; A61N 1/08

(52) U.S. Cl. CPC A61N 1/0452; A61N 1/08; A61N 1/0492

(57) ABSTRACT

A bladder urine volume monitoring system and a bladder urine volume monitoring method are provided. The bladder urine volume monitoring system includes at least one ultrasound patch, at least one muscle stimulation patch, and a control circuit. The ultrasound patch is configured to be attached to a surface of an organism to detect a urine volume in a bladder. The muscle stimulation patch is configured to be attached to the surface of the organism to stimulate a muscle of the bladder. The control circuit is coupled to the ultrasound patch and the muscle stimulation patch. The control circuit drives the ultrasound patch to detect the urine volume in the bladder in a first period. The control circuit drives the muscle stimulation patch to stimulate the muscle of the bladder in a second period.

### CROSS-REFERENCE TO RELATED APPLICATION

This application claims the priority benefits of U.S. provisional application Ser. No. 62/954,631, filed on Dec. 29, 2019, and Taiwan application serial no. 109115361, filed on May 8, 2020. The entirety of each of the above-mentioned patent applications is hereby incorporated by reference herein and made a part of this specification.

### BACKGROUND

### Technical Field

The disclosure relates to a wearable monitoring device and a wearable monitoring system, and in particular, to a bladder urine volume monitoring system and a bladder urine volume monitoring method.

### Description of Related Art

Clinically, most urology patients complain of urgency urination/micturition, frequency urination/micturition, urge urinary incontinence, and nocturia, but no obvious symptoms (e.g., urinary tract infection, bladder stones, etc.) are detected after examination by the doctor. In the absence of symptoms, the doctor may diagnose the condition as an overactive bladder (OAB). The main cause of the overactive bladder is dysfunction of the detrusor muscle of the bladder. For example, even if the bladder is not full (i.e., the urine volume is low), the nerve signal of the bladder tells the brain that the bladder needs to urinate. Therefore, a patient with an overactive bladder frequently feels an urge to urinate, and conditions such as “frequency urination/micturition” or “urgency urination/micturition” occur. The so-called frequency urination/micturition means that the muscle of the bladder is overly active and the frequency of urination exceeds 8 times/day. The so-called urgency urination/micturition means that the bladder muscle contracts earlier than the time point of normal contraction, resulting in a strong urge to urinate.

Due to the conditions such as frequency, urgency urination/micturition, etc. associated with the overactive bladder, patients often feel inconvenient in public areas outside the workplace or home. For example, because of the inconvenience of going to the toilet, patients are afraid to drink water and suffer from mental anxiety resulting from the need to find the toilet at any time, which even leads the patients to go out less frequently. The patients often have low willingness to work, shop, and travel, which creates negative factors in the workplace, social life, family relationships, and mental emotions and consequently seriously affects the quality of life of the patients. Nocturia can also cause conditions such as poor sleep quality and insomnia, which in turn can cause weakness in spirit and physical strength during the day.

Therefore, the monitoring of the urine volume of the bladder is one of the technical issues in this field.

### SUMMARY

The disclosure provides a bladder urine volume monitoring system and a bladder urine volume monitoring method, which can detect a urine volume of the bladder and stimulate the muscle of the bladder.

In an embodiment of the disclosure, the bladder urine volume monitoring system includes at least one ultrasound patch, at least one muscle stimulation patch, and a control circuit. The ultrasound patch is configured to be attached to a surface of an organism to detect a urine volume of a bladder. The muscle stimulation patch is configured to be attached to the surface of the organism to stimulate a muscle of the bladder. The control circuit is coupled to the ultrasound patch and the muscle stimulation patch. The control circuit drives the ultrasound patch to detect the urine volume of the bladder in a first period. The control circuit drives the muscle stimulation patch to stimulate the muscle of the bladder in a second period.

In an embodiment of the disclosure, the bladder urine volume monitoring method includes the following steps. At least one ultrasound patch is attached to a surface of an organism. At least one muscle stimulation patch is attached to the surface of the organism. A control circuit drives the ultrasound patch to detect a urine volume of a bladder in a first period. The control circuit drives the muscle stimulation patch to stimulate a muscle of the bladder in a second period.

Based on the above, in the embodiments of the disclosure, the bladder urine volume monitoring system may use the ultrasound patch to detect the urine volume of the bladder of an organism (e.g., a patient with an overactive bladder), so the urine volume of the organism can be monitored in real time. The bladder urine volume monitoring system may also use the muscle stimulation patch to stimulate the muscle of the bladder, so the abnormal urge to urinate of the organism can be effectively suppressed.

To make the aforementioned more comprehensible, several embodiments accompanied with drawings are described in detail as follows.

### BRIEF DESCRIPTION OF THE DRAWINGS

FIG. 1 is a schematic circuit block view showing a bladder urine volume monitoring system according to an embodiment of the disclosure.

FIG. 2 is a flowchart showing a bladder urine volume monitoring method according to an embodiment of the disclosure.

FIG. 3A to FIG. 3E are schematic views showing an ultrasound probe in FIG. 1 according to an embodiment of the disclosure.

FIG. 4 is a schematic view showing an application scenario of an ultrasound patch and a muscle stimulation patch in FIG. 1 according to an embodiment of the disclosure.

FIG. 5 to FIG. 8 are schematic views showing the ultrasound patch and the ultrasound probe in FIG. 1 according to other different embodiments of the disclosure.

FIG. 9 is a schematic view showing the ultrasound patch and the ultrasound probe in FIG. 1 according to another embodiment of the disclosure.

FIG. 10 is a schematic circuit block view showing a bladder urine volume monitoring system according to another embodiment of the disclosure.

FIG. 11 is a waveform diagram showing a low-frequency voltage emitted by the muscle stimulation patch according to an embodiment of the disclosure.

### DESCRIPTION OF THE EMBODIMENTS

The term “couple or (connect)” used throughout the specification (including the claims) herein may refer to any direct or indirect connection means. For example, if a first device is described to be coupled (or connected) to a second device in the text, it is interpreted that the first device may be directly connected to the second device, or that the first device may be indirectly connected to the second device via other devices or some connection means. The terms “first”, “second”, etc. mentioned throughout the specification (including the claims) herein are used to name the elements or distinguish between different embodiments or ranges, and are not used to limit the upper or lower limit of the number of the element or limit the order of the elements. Moreover, wherever possible, elements/components/steps labeled with the same reference numerals represent the same or similar parts in the drawings and embodiments. Reference may be made between the elements/components/steps labeled with the same reference numerals or described in the same terms in different embodiments for relevant descriptions.

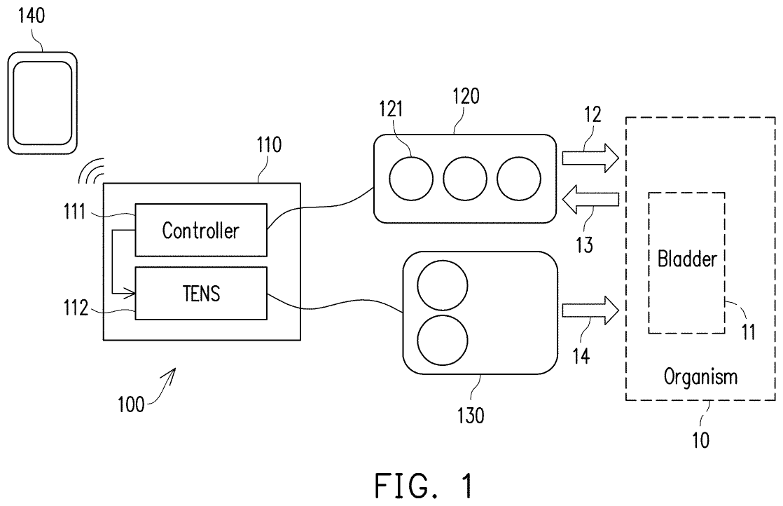

FIG. 1 is a schematic circuit block view showing a bladder urine volume monitoring system 100 according to an embodiment of the disclosure. The bladder urine volume monitoring system 100 includes a control circuit 110, at least one ultrasound patch 120, and at least one muscle stimulation patch 130. The control circuit 110 is coupled to the ultrasound patch 120 and the muscle stimulation patch 130. According to the design requirements, in the embodiment shown in FIG. 1, the control circuit 110 may include a controller 111 and a transcutaneous electrical nerve stimulation (hereinafter referred to as TENS) circuit 112.

According to the application scenario, an organism 10 may be a human being or an animal. For example, the organism 10 may be a patient with an overactive bladder (OAB). The ultrasound patch 120 is configured to be attached to the surface of the organism 10 (e.g., the abdomen of the patient) to emit an ultrasonic wave 12 and collect its reflected wave 13 to thereby detect the urine volume of a bladder 11 of the organism 10. The muscle stimulation patch 130 is configured to be attached to the surface of the organism 10 (e.g., the abdomen of the patient) to emit electrical stimulation energy 14 to stimulate the muscle of the bladder 11.

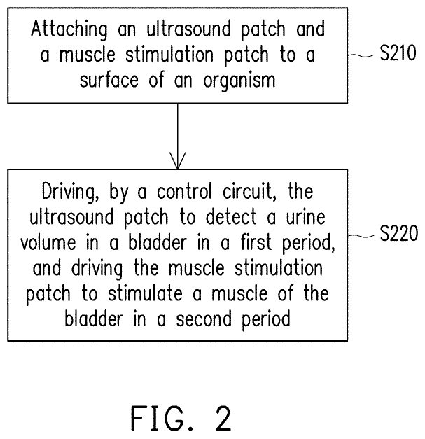

FIG. 2 is a flowchart showing a bladder urine volume monitoring method according to an embodiment of the disclosure. Referring to FIG. 1 and FIG. 2, in step S210, the ultrasound patch 120 and the muscle stimulation patch 130 are attached to the surface of the organism 10. In step S220, the control circuit 110 may drive the ultrasound patch 120 and the muscle stimulation patch 130. For example, the controller 111 of the control circuit 110 may drive the ultrasound patch 120 in a first period to detect the urine volume of the bladder 11. For example, the controller 111 of the control circuit 110 may drive the ultrasound patch 120 to emit an ultrasonic wave 12 and collect its reflected wave 13. Then, the controller 111 may calculate a time difference between the reflected wave 13 and the ultrasonic wave 12 to thereby infer the urine volume in the bladder 11 based on the time difference. Therefore, the urine volume of the organism 10 can be monitored in real time.

According to the design requirements, the ultrasound patch 120 may include one or more ultrasound probes 121. When the ultrasound patch 120 includes a plurality of ultrasound probes 121, the ultrasound probes 121 may be attached to different positions on the surface of the organism 10 near the bladder 11. The controller 111 of the control circuit 110 drives the ultrasound probe 121 (or any of the ultrasound probes 121) in a time-division multiplexing manner to detect the urine volume of the bladder 11. For example, one ultrasound probe 121 may emit an ultrasonic wave 12 to the organism 10. After the ultrasonic wave 12 enters the organism 10, the organism 10 reflects the ultrasonic wave 12. In other words, the organism 10 can provide a reflected wave 13 to the ultrasound patch 120. The urine volume of the bladder 11 can affect the reflected wave 13. Based on the result of detecting the reflected wave 13 by the ultrasound patch 120, the urine volume of the bladder 11 can be monitored or detected. According to the design requirements, in some embodiments, the element which emits the ultrasonic wave 12 and collects the reflected wave 13 may be the same one ultrasound probe 121. In other embodiments, the element which emits the ultrasonic wave 12 and collects the reflected wave 13 may be different ultrasound probes 121.

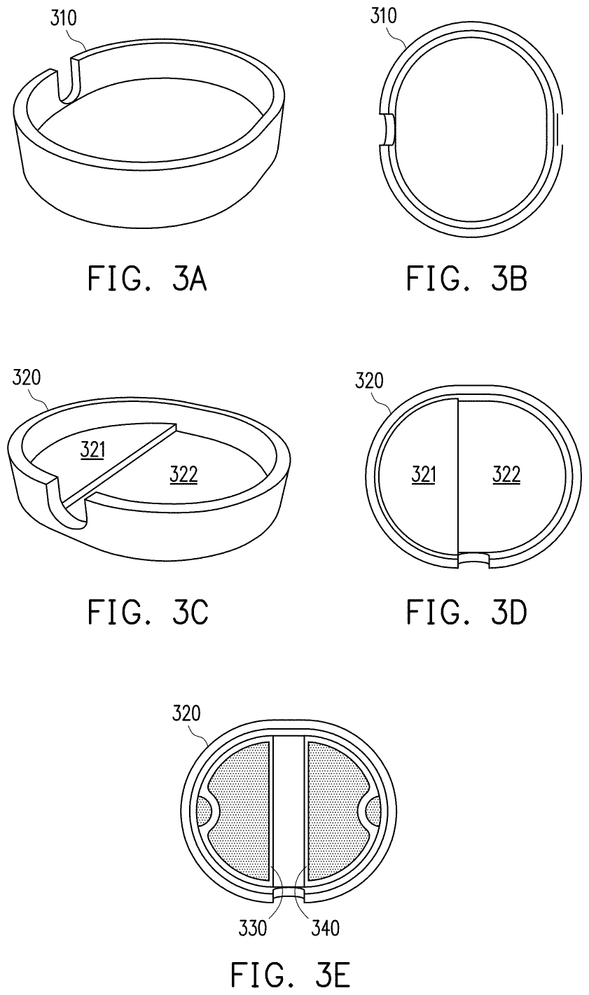

The implementation of the ultrasound probe 121 may be determined according to the design requirements. For example, FIG. 3A to FIG. 3E are schematic structural views showing one single ultrasound probe 121 in FIG. 1 according to an embodiment of the disclosure. In the embodiment shown in FIG. 3A to FIG. 3E, the ultrasound probe 121 includes an upper casing 310, a lower casing 320, a piezoelectric sheet 330, and a piezoelectric sheet 340. The size of ultrasound probe 121 may be determined according to the design requirements.

FIG. 3A is a schematic stereogram view showing the upper casing 310, and FIG. 3B is a schematic top view showing the upper casing 310. In the embodiment shown in FIG. 3A and FIG. 3B, the shape of the upper casing 310 is a quasi-circle. The length of the upper casing 310 (i.e., the long axis of the quasi-circle) may be 23.1 mm, the width of the upper casing 310 (i.e., the short axis of the quasi-circle) may be 20.3 mm, and the height of the upper casing 310 may be 5 mm.

FIG. 3C is a schematic stereogram view showing the lower casing 320, and FIG. 3D is a schematic top view showing the lower casing 320. In the embodiment shown in FIG. 3C and FIG. 3D, the shape of the lower casing 320 is a quasi-circle. The length of the lower casing 320 (i.e., the long axis of the quasi-circle) may be 20.8 mm, the width of the lower casing 320 (i.e., the short axis of the quasi-circle) may be 17.8 mm, and the height of the lower casing 320 may be 4 mm. The bottom plate of the lower casing 320 has an attachment zone 321 and an attachment zone 322.

FIG. 3E is a schematic top view showing the piezoelectric sheet 330 and the piezoelectric sheet 340 disposed on the lower casing 320. In the embodiment shown in FIG. 3E, the piezoelectric sheet 330 and the piezoelectric sheet 340 have a quasi-half-moon shape. The straight side of the piezoelectric sheet 330 faces the straight side of the piezoelectric sheet 340, and a gap is presented between the piezoelectric sheet 330 and the piezoelectric sheet 340. The gap may be defined according to the design requirements. Referring to FIG. 3C, FIG. 3D, and FIG. 3E, the piezoelectric sheet 330 is disposed in the attachment zone 321 of the lower casing 320, and the piezoelectric sheet 340 is disposed in the attachment zone 322 of the lower casing 320. When the upper casing 310 covers the lower casing 320, the piezoelectric sheet 330 and the piezoelectric sheet 340 may be disposed between the upper casing 310 and the lower casing 320. In the embodiment shown in FIG. 3C, FIG. 3D, and FIG. 3E, the normal direction of the attachment zone 321 is different from the normal direction of the attachment zone 322. In other words, the normal direction of the piezoelectric sheet 330 may be different from the normal direction of the piezoelectric sheet 340, or the normal direction of the piezoelectric sheet 330 and the normal direction of the piezoelectric sheet 340 form an acute angle and are not in parallel, so that the emission/reception angles of the ultrasonic wave are different. The normal direction of piezoelectric sheet 330 and the normal direction of piezoelectric sheet 340 may be defined according to the design requirements. In other embodiments, the piezoelectric sheet 330 and the piezoelectric sheet 340 may be substantially on the same plane, and the normal direction of the piezoelectric sheet 330 and the normal direction of the piezoelectric sheet 340 are parallel to each other.

Referring to FIG. 1 and FIG. 3E, the piezoelectric sheet 330 and the piezoelectric sheet 340 may be electrically coupled to the control circuit 110. Based on the driving of the controller 111 of the control circuit 110, the piezoelectric sheet 330 may emit an ultrasonic wave 12. After the piezoelectric sheet 330 emits the ultrasonic wave 12, the piezoelectric sheet 340 may collect a reflected wave 13 corresponding to the ultrasonic wave 12, and return a detection result corresponding to the reflected wave 13 to the controller 111 of the control circuit 110.

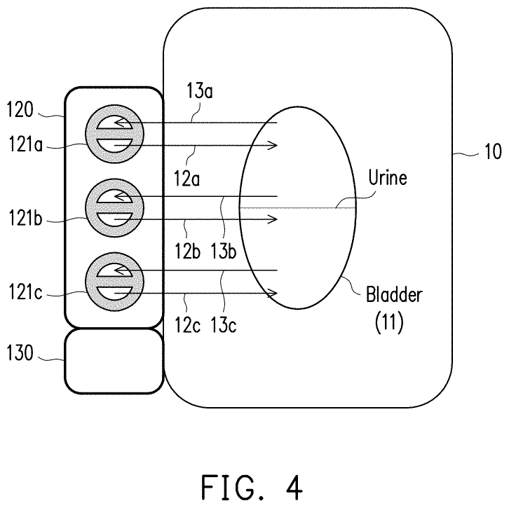

FIG. 4 is a schematic view showing an application scenario of the ultrasound patch 120 and the muscle stimulation patch 130 in FIG. 1 according to an embodiment of the disclosure. It is noted that the ultrasound patch 120 shown in FIG. 4 includes an ultrasound probe 121a, an ultrasound probe 121b, and an ultrasound probe 121c. However, the actual number of the ultrasound patch 120 may be determined according to the design requirements. Furthermore, in other embodiments, the ultrasound probe 121a, the ultrasound probe 121b, and the ultrasound probe 121c may be configured on different ultrasound patches. For description of any one of the ultrasound probe 121a, the ultrasound probe 121b, and the ultrasound probe 121c shown in FIG. 4, reference may be made to the relevant descriptions of FIG. 3A to FIG. 3E, which shall not be repeatedly described herein.

The ultrasound probe 121a may emit an ultrasonic wave 12a and collect a reflected wave 13a. The ultrasound probe 121b may emit an ultrasonic wave 12b and collect a reflected wave 13b. The ultrasound probe 121c may emit an ultrasonic wave 12c and collect a reflected wave 13c. In other words, the ultrasound probes 121a, 121b, and 121c may each independently emit/receive respective signals. For description of any of the ultrasonic wave 12a, the ultrasonic wave 12b, and the ultrasonic wave 12c shown in FIG. 4, reference may be made to the relevant description of the ultrasonic wave 12 shown in FIG. 1. For description of any of the reflected wave 13a, the reflected wave 13b, and the reflected wave 13c shown in FIG. 4, reference may be made to the relevant description of the reflected wave 13 shown in FIG. 1. The ultrasound probes 121 may be attached to different positions on the surface of the organism 10 near the bladder 11, as shown in FIG. 4.

In the first period, the controller 111 of the control circuit 110 may drive the ultrasound probes 121a, 121b, and 121c in a time-division multiplexing manner. For example, the controller 111 may drive the ultrasound probes 121a, 121b, and 121c in a time-division manner and sequentially according to the sequence “from a low position to a high position”. Alternatively, for example, after the ultrasound probe 121a emits the ultrasonic wave 12a and receives the reflected wave 13a, the ultrasound probes 121b and 121c continue to emit/receive the ultrasonic waves. Alternatively, for example, the ultrasound probe 121a emits the ultrasonic wave 12a, and the ultrasound probes 121a, 121b, and 121c collect the reflected wave 13a at the same time. In the next time sequence, the ultrasound probe 121b emits the ultrasonic wave 12b, and the ultrasound probes 121a, 121b, and 121c collect the reflected wave 13b at the same time. Similarly, the ultrasound probe 121c may also operate according to this time-division multiplexing mode. Therefore, the detection results (the reflected waves 13a, 13b, and 13c) of the ultrasound patch 120 can reflect the urine volume of the bladder 11.

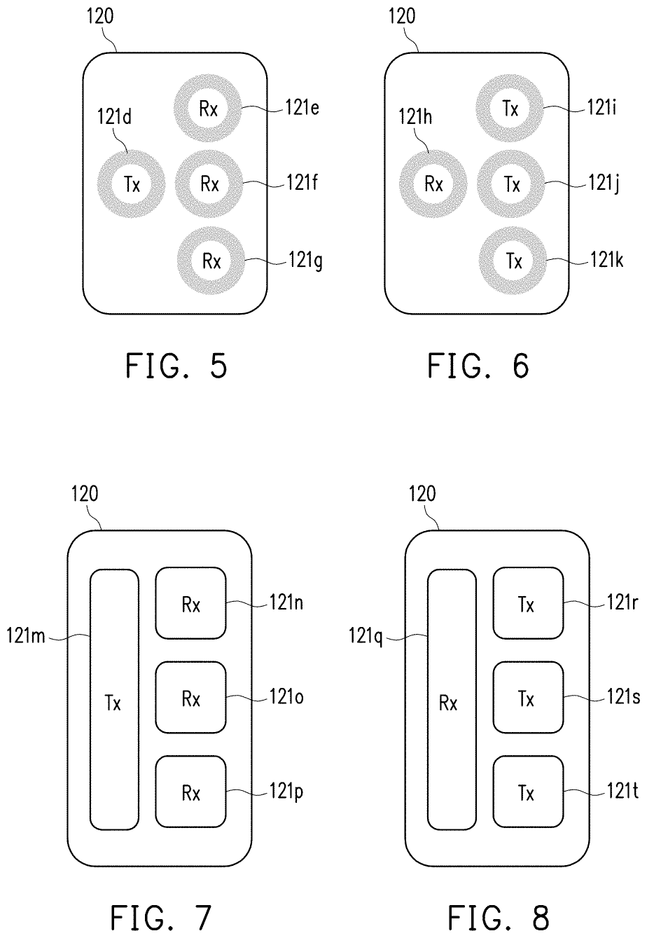

The ultrasound patch 120 and the ultrasound probes 121 shown in FIG. 3A to FIG. 3E and FIG. 4 are only an example of many possible implementations. The implementation of the disclosure is not limited to FIG. 3A to FIG. 3E and FIG. 4. For example, FIG. 5 to FIG. 8 are schematic views showing the ultrasound patch 120 and the ultrasound probe 121 in FIG. 1 according to other different embodiments of the disclosure.

In the embodiment shown in FIG. 5, the ultrasound patch 120 includes an ultrasound probe 121d, an ultrasound probe 121e, an ultrasound probe 121f, and an ultrasound probe 121g. The shape of the ultrasound probes 121d, 121e, 121f, and 121g may be a quasi-circle or another geometric shape. The ultrasound probes 121d, 121e, 121f, and 121g may be disposed at different positions of the ultrasound patch 120, as shown in FIG. 5. According to the design requirements, in other embodiments, the ultrasound probes 121d, 121e, 121f, and 121g may be disposed on different ultrasound patches. The ultrasound probe 121d (emitter) may emit an ultrasonic wave 12, and the ultrasound probes 121e, 121f, and 121g (receivers) may collect a reflected wave 13. The ultrasound probe 121d shown in FIG. 5 may be inferred by referring to the relevant description of the piezoelectric sheet 330 shown in FIG. 3E, and any one of the ultrasound probes 121e, 121f, and 121g shown in FIG. 5 may be inferred by referring to the relevant description of the piezoelectric sheet 340 shown in FIG. 3E.

In the embodiment shown in FIG. 6, the ultrasound patch 120 includes an ultrasound probe 121h, an ultrasound probe 121i, an ultrasound probe 121j, and an ultrasound probe 121k. The shape of the ultrasound probes 121h, 121i, 121j, and 121k may be a quasi-circle or another geometric shape. The ultrasound probes 121h, 121i, 121j, and 121k may be disposed at different positions of the ultrasound patch 120, as shown in FIG. 6. According to the design requirements, in other embodiments, the ultrasound probes 121h, 121i, 121j, and 121k may be disposed on different ultrasound patches. The ultrasound probes 121i, 121j, and 121k (emitters) may emit an ultrasonic wave 12 at different times (time-division multiplexing), and the ultrasound probe 121h (receiver) may collect a reflected wave 13. For example, the ultrasound probe 121i first emits an ultrasonic wave, and the ultrasound probe 121h collects the reflected wave 13. Then, the ultrasound probe 121j emits an ultrasonic wave, and the ultrasound probe 121h collects the reflected wave 13. Next, the ultrasound probe 121k emits an ultrasonic wave, and the ultrasound probe 121h collects the reflected wave 13 to complete a cycle. Alternatively, the three ultrasound probes 121i, 121j, and 121k simultaneously emit ultrasonic waves, and the ultrasound probe 121h collects the reflected wave 13 to complete a cycle. Any one of the ultrasound probes 121i, 121j, and 121k shown in FIG. 6 may be inferred by referring to the relevant description of the piezoelectric sheet 330 shown in FIG. 3E, and the ultrasound probe 121h shown in FIG. 6 may be inferred by referring to the relevant description of the piezoelectric sheet 340 shown in FIG. 3E.

In the embodiment shown in FIG. 7, the ultrasound patch 120 includes an ultrasound probe 121m, an ultrasound probe 121n, an ultrasound probe 121o, and an ultrasound probe 121p. The shape of the ultrasound probes 121m, 121n, 121o, and 121p may be a quasi-rectangle or another geometric shape. The ultrasound probes 121m, 121n, 121o, and 121p may be disposed at different positions of the ultrasound patch 120, as shown in FIG. 7. According to the design requirements, in other embodiments, the ultrasound probes 121m, 121n, 121o, and 121p may be disposed on different ultrasound patches. The ultrasound probe 121m (emitter) may emit an ultrasonic wave 12, and the ultrasound probes 121n, 121o, and 121p (receivers) may collect a reflected wave 13. The ultrasound probe 121m shown in FIG. 7 may be inferred by referring to the relevant description of the piezoelectric sheet 330 shown in FIG. 3E, and any one of the ultrasound probes 121n, 121o, and 121p shown in FIG. 7 may be inferred by referring to the relevant description of the piezoelectric sheet 340 shown in FIG. 3E.

In the embodiment shown in FIG. 8, the ultrasound patch 120 includes an ultrasound probe 121q, an ultrasound probe 121r, an ultrasound probe 121s, and an ultrasound probe 121t. The shape of the ultrasound probes 121q, 121r, 121s, and 121t may be a quasi-rectangle or another geometric shape. The ultrasound probes 121q, 121r, 121s, and 121t may be disposed at different positions of the ultrasound patch 120, as shown in FIG. 8. According to the design requirements, in other embodiments, the ultrasound probes 121q, 121r, 121s, and 121t may be disposed on different ultrasound patches. The ultrasound probes 121r, 121s, and 121t (emitters) may emit an ultrasonic wave 12 at different times (time-division multiplexing), and the ultrasound probe 121q may collect a reflected wave 13. Any one of the ultrasound probes 121r, 121s, and 121t shown in FIG. 8 may be inferred by referring to the relevant description of the piezoelectric sheet 330 shown in FIG. 3E, and the ultrasound probe 121q (receiver) shown in FIG. 8 may be inferred by referring to the relevant description of the piezoelectric sheet 340 shown in FIG. 3E.

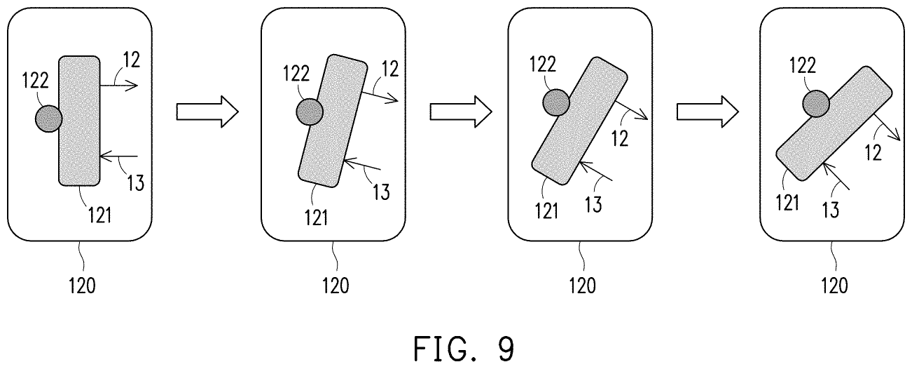

FIG. 9 is a schematic view showing the ultrasound patch 120 and the ultrasound probe 121 in FIG. 1 according to another embodiment of the disclosure. In the embodiment shown in FIG. 9, the ultrasound patch 120 may include a housing for accommodating the ultrasound probe 121. The ultrasound probe 121 shown in FIG. 9 may be inferred by referring to the relevant descriptions of FIG. 3A to FIG. 3E. FIG. 9 shows a side view of the ultrasound probe 121 in FIG. 3A to FIG. 3E. The side view of FIG. 3A to FIG. 3E is a rectangle, so the ultrasound probe 121 is shown as a rectangle in FIG. 9. The normal direction of the upper casing 310 (or the piezoelectric sheets 330 and 340) of the ultrasound probe 121 is substantially the same as (parallel to) the direction of the ultrasonic wave 12 shown in FIG. 9 and/or the direction of the reflected wave 13 shown in FIG. 9. In other words, referring to FIG. 3A to FIG. 3E and FIG. 9, the ultrasonic wave 12 emitted from the piezoelectric sheet 330 disposed in the ultrasound probe 121 can pass through the upper casing 310 and reach the bladder, and the piezoelectric sheet 340 disposed in the ultrasound probe 121 may sense the reflected wave 13 passing through the upper casing 310. The ultrasound probe 121 is pivotally disposed in the housing of the ultrasound patch 120. In other words, the ultrasound probe 121 is disposed on a rotating shaft 122. FIG. 9 shows that the ultrasound probe 121 changes the emission/reception angle as the rotating shaft 122 rotates, and the rotation of the rotating shaft 122 may be set to be a reciprocating movement within an arc angle interval. Based on the control of the control circuit 110, the direction of the ultrasound probe 121 may be changed. Therefore, the ultrasound probe 121 shown in FIG. 9 can scan the entire bladder 11 so that the control circuit 110 can learn the urine volume of the bladder 11.

Referring to FIG. 1 and FIG. 2, in a second period different from the first period (i.e., the period of driving the ultrasound patch 120), the TENS circuit 112 of the control circuit 110 may drive the muscle stimulation patch 130 to stimulate the muscle of the bladder 11 (step S220). Therefore, the abnormal urge to urinate of the organism 10 can be effectively suppressed. This embodiment does not limit the implementation of the TENS circuit 112. According to the design requirements, the TENS circuit 112 may be a general TENS circuit or another TENS circuit.

For example, the control circuit 110 may selectively drive the muscle stimulation patch 130 according to the urine volume in the bladder 11 to suppress abnormal contraction of the detrusor muscle of the bladder 11. When the urine volume in the bladder 11 has not reached a normal urination threshold, the TENS circuit 112 of the control circuit 110 may drive the muscle stimulation patch 130 to suppress the abnormal contraction of the detrusor muscle of the bladder 11 and reduce the urgency of urination. Therefore, the abnormal urge to urinate of the organism 10 can be effectively suppressed. The “normal urination threshold” may be determined according to the design requirements. When the urine volume in the bladder 11 reaches (or exceeds) the normal urination threshold, the TENS circuit 112 of the control circuit 110 may suspend driving the muscle stimulation patch 130. At this time, the detrusor muscle of the bladder 11 can contract normally and cause the organism 10 to have an urge to urinate.

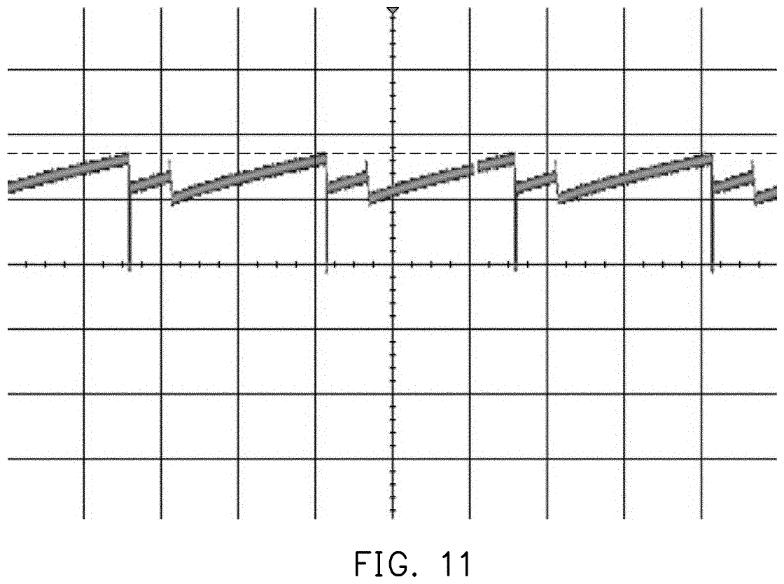

This embodiment does not limit the method and details of stimulating the muscle of the bladder 11 by the muscle stimulation patch 130. For example, according to the design requirements, the stimulation method (i.e., the stimulation to the muscle of the bladder 11 by the muscle stimulation patch 130) for suppressing an abnormal urge to urinate may include the general electrical stimulation or other muscle stimulation techniques. For example, based on the driving of the control circuit 110, the muscle stimulation patch 130 may emit a low-frequency voltage to electrically stimulate the muscle of the bladder 11. FIG. 11 is a waveform diagram showing a low-frequency wave emitted by the muscle stimulation patch 130 according to an embodiment of the disclosure. The horizontal axis shown in FIG. 11 represents time (second), and the vertical axis represents the voltage level (volt) of the ultrasonic wave. FIG. 11 shows a sawtooth wave with mixed oblique long sides and oblique short sides, and the two alternately stimulate to exhibit low periodic or low frequency stimulation. The oblique long side or the oblique short side each shows an increasing voltage, and the upper and lower voltage limits of the oblique long side and the oblique short side are within a specific interval. In this embodiment, the peak value is 92 V and the frequency is 1.9 Hz. The control circuit 110 can effectively monitor the bladder 11 and work/cooperate with the muscle stimulation patch 130 to suppress the urge to urinate and/or perform a physical therapy on the bladder 11.

On the other hand, the physical therapy performed by the muscle stimulation patch 130 may also strengthen the pelvic floor muscles and/or strengthen the urethral sphincter. The strengthened urethral sphincter may delay the urination time to mitigate urinary continence and reduce the effect of the overactive bladder.

According to the design requirements and/or application requirements, the bladder urine volume monitoring system 100 may further include a portable electronic device 140. This embodiment does not limit the implementation of the portable electronic device 140. For example, the portable electronic device 140 may be a smartphone, a tablet computer, a notebook computer, a personal digital assistant (PDA), or another computing device. The portable electronic device 140 may establish a communicative connection with the control circuit 110. According to the design requirements, in some embodiments, the communicative connection may include any wireless communication network.

Referring to FIG. 1, when a communicative connection has been established between the portable electronic device 140 and the control circuit 110, the portable electronic device 140 may control the control circuit 110 so that the ultrasound patch 120 emits an ultrasonic wave 12 and obtains a reflected wave 13. Based on the reflected wave 13, the portable electronic device 140 can calculate the urine volume in the bladder 11. When the urine volume in the bladder 11 reaches the normal urination threshold, the portable electronic device 140 may notify the user. The user may perform personalized long-term monitoring through the portable electronic device 140, and the portable electronic device 140 may display bladder volume information in real time to allow the user to know when to urinate. In addition, the portable electronic device 140 may perform learning analysis based on the urine volume record of the organism 10, and provide a stimulation therapy through the muscle stimulation patch 130 to stimulate the autonomic nerves and perform autonomous bladder training, thereby reducing muscle sensitivity of the bladder and easing mental anxiety.

According to different application scenarios, the user may be the same as (or different from) the organism 10. For example, in a single-person application scenario, the user may be the same as the organism 10. For example, the user may be a patient. Therefore, the patient can know the real-time urine volume of his or her bladder 11 at any time. When the urine volume in the bladder 11 reaches the normal urination threshold, the portable electronic device 140 may notify the patient. In an application scenario where a caregiver takes care of a patient, the user may be different from the organism 10. In other words, the user may be the caregiver and the organism 10 may be the patient (or animal) being taken care of. Therefore, the caregiver can know the real-time urine volume of the bladder 11 of the patient (or animal) at any time. When the urine volume in the bladder 11 reaches the normal urination threshold, the portable electronic device 140 may notify the caregiver.

According to the design requirements and/or application requirements, the bladder urine volume monitoring system 100 may include more functions. For example, in the scenario where the organism 10 (patient) goes out, when the portable electronic device 140 notifies the user, the portable electronic device 140 may further provide toilet location information to the user. As another example, in other embodiments, it is assumed that the portable electronic device 140 may detect the movement of the organism 10. When the organism 10 is in a non-moving state, the portable electronic device 140 may monitor the urine volume through the control circuit 110 (step S220 in FIG. 2) to selectively detect the urine volume of the bladder 11 and/or selectively stimulate the muscle of the bladder 11. When the organism 10 is in a moving state, the portable electronic device 140 may suspend the monitoring of the urine volume.

In some embodiments, the portable electronic device 140 may collect personal behavior information through long-term monitoring and perform personalized behavior analysis and determination, thereby self-adjusting the model parameters (e.g., a bladder volume parameter and/or other parameters) and estimating the time course of urination. In other embodiments, the personalized behavior analysis of the portable electronic device 140 may be operated with the muscle stimulation patch 130 (e.g., a low-frequency electrical stimulation therapy module) to perform a comprehensive therapy. For example, the portable electronic device 140 and/or the control circuit 110 may stimulate the autonomic nerves of the organism 10 through the muscle stimulation patch 130 to perform an autonomous bladder training, so as to reduce muscle sensitivity of the bladder and ease mental anxiety, and thereby delaying the urination time. The portable electronic device 140 may switch the electrical stimulation mode of the muscle stimulation patch 130 according to different requirements.

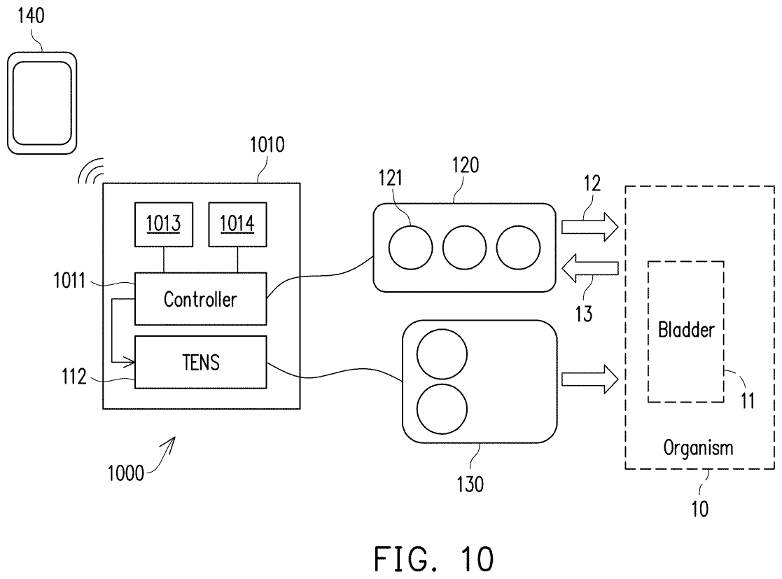

FIG. 10 is a schematic circuit block view showing a bladder urine volume monitoring system 1000 according to another embodiment of the disclosure. The relevant description of FIG. 2 is also applicable to the bladder urine volume monitoring system 1000 shown in FIG. 10. The bladder urine volume monitoring system 1000 includes a control circuit 1010, at least one ultrasound patch 120, and at least one muscle stimulation patch 130. According to the design requirements and/or application requirements, the bladder urine volume monitoring system 1000 may further include a portable electronic device 140. The control circuit 1010 is coupled to the ultrasound patch 120 and the muscle stimulation patch 130. According to the design requirements, in the embodiment shown in FIG. 10, the control circuit 1010 may include a controller 1011 and a TENS circuit 112. For descriptions of the organism 10 and the bladder 11 shown in FIG. 10, reference may be made to the relevant descriptions of the organism 10 and the bladder 11 shown in FIG. 1 or FIG. 4. For description of the ultrasound patch 120 shown in FIG. 10, reference may be made to the relevant descriptions of the ultrasound patch 120 shown in FIG. 1, FIG. 3A to FIG. 3E, FIG. 4, FIG. 5, FIG. 6, FIG. 7, FIG. 8, and FIG. 9. For descriptions of the control circuit 1010, the muscle stimulation patch 130, and the portable electronic device 140 shown in FIG. 10, reference may be made to the relevant descriptions of the control circuit 110, the muscle stimulation patch 130, and the portable electronic device 140 shown in FIG. 1. The relevant descriptions shall not be repeated herein.

In the embodiment shown in FIG. 10, the control circuit 1010 may further include a micro vibration motor 1013 and/or an accelerometer 1014. The micro vibration motor 1013 may be disposed on the surface of the organism 10. When the urine volume in the bladder 11 reaches the normal urination threshold, the controller 1011 of the control circuit 1010 may drive the micro vibration motor 1013 so as to notify the organism 10 through vibration. For example, when the communicative connection between the portable electronic device 140 and the control circuit 1010 is not established, the control circuit 1010 may independently perform urine volume monitoring, muscle stimulation, notification, and/or other operations. When the portable electronic device 140 is not connected, the controller 1011 may send a vibration signal to the organism 10 (user) through the micro vibration motor 1013 to remind the organism 10 to urinate (which eases urination urgency and psychological emotions).

The accelerometer 1014 is disposed on the surface of the organism 10. The accelerometer 1014 may detect the movement of the organism 10. For example, the accelerometer 1014 may detect the state (e.g., stationary, walking, etc.) of the organism 10. When the organism 10 is in a non-moving state (e.g., a stationary state), the controller 1011 of the control circuit 1010 may monitor the urine volume (step S220) to selectively detect the urine volume of the bladder 11 and/or selectively stimulate the muscle of the bladder 11. When the organism 10 is in a moving state, the controller 1011 of the control circuit 1010 may suspend the monitoring of the urine volume.

In some application scenarios, the control circuit 1010 may return the detection result of the accelerometer 1014 to the portable electronic device 140. The portable electronic device 140 may send a control signal to the control circuit 1010 according to the detection result of the accelerometer 1014 to drive the ultrasound patch 120 to emit an ultrasonic wave 12. For example, when the organism 10 is in the stationary state, the portable electronic device 140 may command the control circuit 1010 to monitor the urine volume (step S220 in FIG. 2). The control circuit 1010 may return the detection result (the reflected wave 13) of the ultrasound patch 120 to the portable electronic device 140. Based on the detection result (the reflected wave 13) of ultrasound patch 120, the portable electronic device 140 can calculate the liquid volume in the bladder 11. The portable electronic device 140 may send a reminder according to the urine volume in the bladder 11 to remind the user. The portable electronic device 140 may also determine whether to monitor the urine volume according to the urine volume in the bladder 11 (step S220).

According to different design requirements, the blocks including the control circuit 110, the controller 111, the control circuit 1010, the controller 1011, and/or the TENS circuit 112 may be implemented as hardware, firmware, software (i.e., programs), or combinations thereof.

In the form of hardware, the blocks including the control circuit 110, the controller 111, the control circuit 1010, the controller 1011, and/or the TENS circuit 112 may be implemented as logic circuits on integrated circuits. The relevant functions of the control circuit 110, the controller 111, the control circuit 1010, the controller 1011, and/or the TENS circuit 112 may be implemented as hardware by using hardware description languages (e.g., Verilog HDL or VHDL) or other suitable programming languages. For example, the relevant functions of the control circuit 110, the controller 111, the control circuit 1010, the controller 1011, and/or the TENS circuit 112 may be implemented in one or more controllers, microcontrollers, microprocessors, application-specific integrated circuits (ASIC), digital signal processors (DSP), field programmable gate arrays (FPGA), and/or various other logic blocks, modules, and circuits in processing units.

In the form of software and/or firmware, the relevant functions of the control circuit 110, the controller 111, the control circuit 1010, the controller 1011, and/or the TENS circuit 112 may be implemented as programming codes. For example, the control circuit 110, controller 111, the control circuit 1010, the controller 1011, and/or the TENS circuit 112 may be implemented by using general programming languages (e.g., C, C++, or combined languages) or other suitable programming languages. The programming codes may be recorded/stored in a recording medium, and the recording medium includes, for example, a read only memory (ROM), a storage device, and/or a random access memory (RAM). A computer, a central processing unit (CPU), a controller, a microcontroller, or a microprocessor may read and execute the programming codes in the recording medium so as to achieve the relevant functions. As the recording medium, a “non-transitory computer readable medium” may be used. For example, a tape, a disk, a card, a semiconductor memory, a programmable logic circuit, etc. may be used. Moreover, the program may also be provided to the computer (or the CPU) via any transmission medium (a communication network, a broadcast wave, etc.). The communication network is, for example, the Internet, wired communication, wireless communication, or other communication media.

In summary of the above, in the embodiments, the bladder urine volume monitoring system may use the ultrasound patch 120 to detect the urine volume of the bladder 11 of the organism 10 (e.g., a patient with an overactive bladder), so the urine volume of the organism 10 can be monitored or detected in real time. In some embodiments, the bladder urine volume monitoring system may perform personalized behavior analysis based on the history record of the urine volume of the bladder 11. In addition, the bladder urine volume monitoring system may also use the muscle stimulation patch 130 to stimulate the muscle of the bladder 11, so that the abnormal urge to urinate of the organism 10 can be effectively suppressed. In some embodiments, the personalized behavior analysis of the bladder urine volume monitoring system may be operated with the muscle stimulation patch 130 (e.g., a low-frequency electrical stimulation therapy module) to perform a comprehensive therapy. The bladder urine volume monitoring system may switch the electrical stimulation mode of the muscle stimulation patch 130 according to different requirements. For example, the bladder urine volume monitoring system may stimulate the autonomic nerves of the organism 10 through the muscle stimulation patch 130 to perform an autonomous bladder training, so as to reduce muscle sensitivity of the bladder and ease mental anxiety, and thereby delaying the urination time.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A bladder urine volume monitoring system comprising:
at least one ultrasound patch configured to be attached to a surface of an organism to detect a urine volume of a bladder;
at least one muscle stimulation patch configured to be attached to the surface of the organism to stimulate a muscle of the bladder; and
a control circuit coupled to the at least one ultrasound patch and the at least one muscle stimulation patch, and configured to drive the at least one ultrasound patch to detect the urine volume of the bladder in a first period and drive the at least one muscle stimulation patch to stimulate the muscle of the bladder in a second period.

2. The bladder urine volume monitoring system according to claim 1, wherein the at least one ultrasound patch comprises a plurality of ultrasound probes, wherein the ultrasound probes are adapted to be attached to different positions on the surface near the bladder, and the control circuit drives any one of the ultrasound probes in a time-division multiplexing manner to detect the urine volume of the bladder.

3. The bladder urine volume monitoring system according to claim 1, wherein the at least one ultrasound patch comprises an ultrasound probe, and the ultrasound probe comprises:
an upper casing;
a lower casing;
a first piezoelectric sheet disposed between the upper casing and the lower casing and configured to emit an ultrasonic wave based on driving of the control circuit; and
a second piezoelectric sheet disposed between the upper casing and the lower casing and located on a same plane as the first piezoelectric sheet, and configured to collect a reflected wave corresponding to the ultrasonic wave and return a detection result corresponding to the reflected wave to the control circuit.

4. The bladder urine volume monitoring system according to claim 3, wherein the first piezoelectric sheet and the second piezoelectric sheet have a quasi-half-moon shape, a straight side of the first piezoelectric sheet faces a straight side of the second piezoelectric sheet, and a gap is presented between the first piezoelectric sheet and the second piezoelectric sheet.

5. The bladder urine volume monitoring system according to claim 1, wherein the at least one ultrasound patch comprises an ultrasound probe, and the ultrasound probe comprises:
an upper casing;
a lower casing;
a first piezoelectric sheet disposed between the upper casing and the lower casing and configured to emit an ultrasonic wave based on driving of the control circuit; and
a second piezoelectric sheet disposed between the upper casing and the lower casing and configured to collect a reflected wave corresponding to the ultrasonic wave and return a detection result corresponding to the reflected wave to the control circuit, wherein a normal direction of the first piezoelectric sheet is different from a normal direction of the second piezoelectric sheet.

6. The bladder urine volume monitoring system according to claim 1, wherein the at least one muscle stimulation patch emits a low-frequency wave based on driving of the control circuit to stimulate the muscle of the bladder.

7. The bladder urine volume monitoring system according to claim 1, wherein the control circuit selectively drives the at least one muscle stimulation patch according to the urine volume in the bladder so as to suppress abnormal contraction of a detrusor muscle of the bladder.

8. The bladder urine volume monitoring system according to claim 1, wherein the control circuit comprises:
a micro vibration motor disposed on the surface of the organism, wherein when the urine volume in the bladder reaches a normal urination threshold, the control circuit drives the micro vibration motor to notify the organism.

9. The bladder urine volume monitoring system according to claim 1, wherein the control circuit comprises:
an accelerometer disposed on the surface of the organism and adapted to detect a movement of the organism, wherein
when the organism is in a non-moving state, the control circuit performs a urine volume monitoring to selectively detect the urine volume of the bladder or selectively stimulate the muscle of the bladder, and
when the organism is in a moving state, the control circuit suspends the urine volume monitoring.

10. The bladder urine volume monitoring system according to claim 1, further comprising:
a portable electronic device configured to establish a communicative connection with the control circuit, wherein the portable electronic device controls the control circuit to cause the at least one ultrasound patch to emit an ultrasonic wave and obtain a reflected wave corresponding to the ultrasonic wave, to calculate the urine volume in the bladder based on the reflected wave.

11. The bladder urine volume monitoring system according to claim 10, wherein when the urine volume in the bladder reaches a normal urination threshold, the portable electronic device notifies a user.

12. The bladder urine volume monitoring system according to claim 11, wherein when the portable electronic device notifies the user, the portable electronic device further provides toilet location information to the user.

13. The bladder urine volume monitoring system according to claim 10, wherein
the portable electronic device is adapted to detect a movement of the organism,
when the organism is in a non-moving state, the portable electronic device performs a urine volume monitoring to selectively detect the urine volume of the bladder or selectively stimulate the muscle of the bladder, and
when the organism is in a moving state, the portable electronic device suspends the urine volume monitoring.

14. A bladder urine volume monitoring method comprising:
attaching at least one ultrasound patch and at least one muscle stimulation patch to a surface of an organism;
driving, by a control circuit, the at least one ultrasound patch to detect a urine volume of a bladder in a first period; and
driving, by the control circuit, the at least one muscle stimulation patch to stimulate a muscle of the bladder in a second period.

15. The bladder urine volume monitoring method according to claim 14, wherein the at least one ultrasound patch comprises a plurality of ultrasound probes, the ultrasound probes are adapted to be attached to different positions on the surface near the bladder, and the bladder urine volume monitoring method further comprises:
driving, by the control circuit, any one of the ultrasound probes in a time-division multiplexing manner to detect the urine volume of the bladder.

16. The bladder urine volume monitoring method according to claim 14, further comprising:
emitting, by the at least one muscle stimulation patch, a low-frequency wave based on driving of the control circuit to stimulate the muscle of the bladder.

17. The bladder urine volume monitoring method according to claim 14, further comprising:
selectively driving, by the control circuit, the at least one muscle stimulation patch according to the urine volume in the bladder so as to suppress abnormal contraction of a detrusor muscle of the bladder.

18. The bladder urine volume monitoring method according to claim 14, further comprising:
driving, by the control circuit, a micro vibration motor to notify the organism when the urine volume in the bladder reaches a normal urination threshold.

19. The bladder urine volume monitoring method according to claim 14, further comprising:
detecting, by an accelerometer, a movement of the organism;
performing, by the control circuit, a urine volume monitoring to selectively detect the urine volume of the bladder or selectively stimulate the muscle of the bladder when the organism is in a non-moving state; and
suspending, by the control circuit, the urine volume monitoring when the organism is in a moving state.

20. The bladder urine volume monitoring method according to claim 14, further comprising:
establishing a communicative connection between a portable electronic device and the control circuit;
controlling, by the portable electronic device, the control circuit to cause the at least one ultrasound patch to emit an ultrasonic wave and obtain a reflected wave corresponding to the ultrasonic wave; and
calculating, by the portable electronic device, the urine volume in the bladder based on the reflected wave.

21. The bladder urine volume monitoring method according to claim 20, further comprising:
notifying a user by the portable electronic device when the urine volume in the bladder reaches a normal urination threshold.

22. The bladder urine volume monitoring method according to claim 21, further comprising:
providing, by the portable electronic device, toilet location information to the user when the portable electronic device notifies the user.

23. The bladder urine volume monitoring method according to claim 20, further comprising:
detecting, by the portable electronic device, a movement of the organism;
performing, by the portable electronic device, a urine volume monitoring to selectively detect the urine volume of the bladder or selectively stimulate the muscle of the bladder when the organism is in a non-moving state; and
suspending, by the portable electronic device, the urine volume monitoring when the organism is in a moving state.
